(19) United States
(12) Patent Application Publication
(10) Pub. No.: US 2005/0008712 A1
(43) Pub. Date: Jan. 13, 2005

(54) Compositions incorporating high-caffeine green tea extract and related methods for promoting healthy body weight

(76) Inventors: Debasis Bagchi, Concord, CA (US); Shil C. Kothari, Fairfield, CA (US)

Correspondence Address: SHEPPARD, MULLIN, RICHTER & HAMPTON LLP, 333 SOUTH HOPE STREET, 48TH FLOOR, LOS ANGELES, CA, 90071-1448, US

(21) Appl. No.: 10/615,887

(22) Filed: Jul. 8, 2003

(51) Int. Cl. A61K 35/78; A61K 31/522; A61K 31/7048; A61K 31/353

(52) U.S. Cl. 424/729; 514/27; 514/263.31; 514/456

(57) ABSTRACT

Methods for promoting healthy body weight, increasing energy level and improving a variety of related physiological factors include administering to those persons or other mammals effective amounts of a composition incorporating EGCG and caffeine derived from green tea, preferably in equal or nearly equal amounts. The EGCG and caffeine in these amounts work synergistically to further promote healthy body weight, increase energy level and improve these physiological factors.

### FIELD OF THE INVENTION

The present invention generally relates to providing a method or compound for promoting healthy body weight, increasing energy level and improving a variety of related physiological factors and, more particularly, to providing a composition that includes effective amounts of EGCG and caffeine derived from green tea to further promote healthy body weight, increase energy level and improve a variety of related physiological factors and a method of administering the composition.

### BACKGROUND OF THE INVENTION

Excess body weight is becoming more prevalent worldwide at an alarming rate, both in developing and developed countries. Approximately 61 percent of adults in the U.S. are overweight (i.e., having a body mass index (BMI) of greater than 25 kg/m²), while more than 26 percent of U.S. adults are obese (i.e., having a BMI of greater than 30 kg/m²). Obesity is the second leading cause of premature death in the U.S. Approximately 300,000 Americans die each year from complications caused by obesity. According to the World Health Organization, there are over 300 million obese adults worldwide. Environmental and behavioral changes brought about by economic development, modernization and urbanization have been linked to the global rise in obesity in adults and children, the true health consequences of which may not be fully known for years to come. Consumption of western-style diets, low levels of physical activity and sedentary lifestyles generally have been implicated in the worldwide trend of weight gain.

Increase in body weight results from an imbalance between energy intake and expenditure in a person, manifested by excessive expansion of adipose tissue mass in the person. Obesity leads to a number of poor health factors. In particular, obesity increases the risk of high blood pressure, hypertension, type II diabetes, arthritis, elevated cholesterol, and cancer. Although 30-40% of obese people claim they are trying to lose or maintain body weight, their success rate is low. Dietary approaches for the management of excess body weight have been unsuccessful due to improper caloric restriction and/or lack of physical exercise. Low calorie diets can provide for temporary weight loss, but they have not proven themselves as long-term solutions for people trying to lose or maintain weight. Drugs that suppress appetite, reduce food intake, increase energy expenditure and/or affect nutrient partitioning or metabolism have potential efficacy in reducing body weight. Unfortunately, these also frequently are accompanied by adverse side effects, some of which are life threatening.

High blood cholesterol, high blood triglyceride levels, and obesity all are indicators of increased risk for heart disease and other health maladies. In particular, high levels of total cholesterol, LDL cholesterol or triglycerides, as well as low levels of HDL cholesterol, all are risk factors for various cardiovascular diseases. These conditions are exacerbated by many factors, including poor diet, lack of exercise and obesity. Prevalence for obesity can be reflected in excessive eating and also by genetic factors. One method for reducing appetite, and therefore excessive eating, is by raising serotonin levels in a person. Increased brain levels of serotonin, an important neurotransmitter involved in proper brain function, including regulation of sleep and mood, have also been linked with appetite suppression. Also, a known biomarker for genetic propensity of a person toward obesity is serum leptin, a hormone encoded by the gene that regulates body weight. Leptin binds to receptors in the brain, where it activates signals that inhibit food intake and increase energy expenditure. Studies have shown that plasma leptin levels are higher in overweight than in non-overweight individuals, and higher in women than in men.

The methods described above to treat obesity in humans may be applicable to treating other mammals as well, including animals commonly kept as pets, such as dogs and cats. Excess body weight has reached epidemic proportions in, and is the most common nutritional disorder among, pets. It is estimated that 50% of pets (or roughly 60 million animals) in the United States are overweight or obese (a weight ten percent over ideal body weight is considered overweight, and a weight twenty percent over ideal body weight is clinically defined as obese). An extra five pounds on a dog that should weigh 17 pounds or an extra three pounds on a cat that should weight 10 pounds is comparable to an extra 50 pounds on a person who should weigh 170 pounds. Overweight pets are at higher risk of developing health problems such as heart disease, skeletal problems, breathing problems, diabetes and arthritis. Traditionally, weight management in veterinary medicine relies on one or more recommendations. A veterinarian may prescribe high fiber/reduced calorie diets, or advocate other dietary changes focusing on a decrease in overall caloric intake. Another method to manage pet weight is to increase exercise. Untreated obesity can be a devastating condition for a pet, and instituting an obesity-management program will add quality years to a pet's life.

Various methods exist for treating obesity and the other related health factors discussed above, such as improved diet, increased exercise, and various medications. These, however, have not been entirely effective treatments. Diet modification and increased exercise can be difficult for some individuals to maintain for an extended period, and medications introduce the possibility of negative side effects.

A variety of biological factors are relevant to regulation of body weight in persons. One of these is leptin level. Leptin is a 167 amino acid protein hormone encoded by the gene that regulates body weight. Synthesized and secreted by adipocytes (fat cells), leptin binds to receptors in the brain, where it activates signals that inhibit food intake and increase energy expenditure. When receptor-binding activity is diminished, a condition called “leptin resistance,” plasma leptin levels increase and the leptin loses its ability to inhibit food intake and increase energy expenditure. Studies have shown that plasma leptin levels are higher in overweight than in non-overweight individuals, and higher in women than in men. Leptin is synthesized and secreted by adipocytes, is present in the bloodstream in amounts related to the amount of fat in the body, and acts primarily on the brain to regulate food intake. Leptin has been shown to be able to modulate insulin secretion and action through these receptors. These findings confirm earlier observations of higher leptin levels in obese individuals than in lean individuals.

Another important factor in regulating body weight is fat metabolysis. Fat metabolites are products of fat degradation. Following exercise or other fat “burning” processes, fat tissue breaks down into small molecular components, including malondialdehyde, formaldehyde, acetaldehyde and acetone. Increased urinary levels of fat metabolites indicates increased fat degradation or “burning,” which can result in weight loss.

Also, serum serotonin level affects eating behavior and body weight. Increased plasma levels of serotonin are associated with decreased food intake, reduced weight gain and increased energy expenditure. Because serotonin has been implicated in the regulation of eating behavior and body weight regulation, appetite suppression could be mediated by this serotonin.

One chemical known to promote weight loss is caffeine. Caffeine, or trimethylxanthine (C₈H₁₀N₄O₂), acts as a mild stimulant to the central nervous system when ingested by persons in amounts up to about 300 mg. Higher levels of caffeine consumption can lead to headache, nervousness and loss of sleep. Caffeine also exhibits effects that indicate it may serve as a mild appetite suppressant. Additionally, caffeine is believed to increase metabolism and bum calories by a process known as “thermogenesis,” and it also acts as a diuretic to remove excess fluids from a person. Common sources of caffeine include coffee and tea. Despite its weight loss properties, caffeine often is removed from these products prior to consumption, as people seek to reduce caffeine consumption for health reasons.

Besides caffeine, tea, and particularly green tea, includes polyphenols having a variety of known beneficial effects. In particular, the main polyphenol of green tea is (−)-epigallo-catechin-3-gallate (EGCG). EGCG is a potent antioxidant and anti-neoplastic agent. Green tea catechins such as EGCG have been shown to inhibit lipid oxidation in low-density lipoprotein and provide a variety of antioxidant effects. Green tea incorporating EGCG also is known to possess thermogenic properties, increasing energy expenditure and fat oxidation in humans. In particular, it plays a role in the control of body composition via sympathetic activation of thermogenic fat oxidation. A previous study showed that relative placebo treatment with green tea extract resulted in a 4% increase in 24-hour Energy Expenditure and a significant decrease in 24-hour Respiratory Quotient from 0.88 to 0.85, without any change in urinary nitrogen. Twenty-four hour urinary norepinephrine excretion was higher during treatment with the green tea extract than with the placebo by an average of 40%. For comparison, treatment with caffeine in amounts equivalent to those naturally occurring in the green tea extract had no significant effect on any of these measured factors.

Another study demonstrated that EGCG significantly reduces food intake, body weight, blood cholesterol and triglycerides in rats. For clear assessment of the possible physiological effects of green tea consumption, the rats were injected with pure green tea catechin to study its effects on the endocrine systems of the rats. EGCG was found to significantly reduced food intake and body weight in the rats. Similar effects were observed in both lean and obese male Zucker rats, suggesting that the effect of EGCG was independent of an intact leptin receptor. EGCG may interact specifically with a component of a leptin (i.e., the so-called “obesity gene”) independent of its appetite control pathway. These researchers also have shown that EGCG injected into rats caused the animals to lose 20% of their body weight after daily injections for a week.

Both caffeine and EGCG are known to exhibit some weight control properties in persons or other mammals consuming them. However, conventional administration of these does not provide weight loss that is fully satisfactory. It is apparent from the above that a need exists for improved methods and compositions for controlling body weight and improving the health condition of persons or other mammals prone to excess body weight, including improvement of body mass index (an indicator of healthy body weight), serum leptin levels, and the cardiovascular risk factors total cholesterol, LDL cholesterol, HDL cholesterol and triglycerides. The present invention fulfills this need and provides further related advantages.

### SUMMARY OF THE INVENTION

The present invention resides in an improved method and composition for promoting healthy body weight, increasing energy level and improving a variety of related physiological factors. The method and composition of the present invention utilizes amounts of EGCG and caffeine that work synergistically to further promote healthy body weight, increase energy level and improve various physiological factors.

In a more detailed feature of the invention, the method of the present invention includes administering to those persons or other mammals effective amounts of a composition incorporating EGCG and caffeine derived from green tea.

In yet another more detailed feature of the invention, the composition of the present invention comprises EGCG and caffeine in a ratio by weight between 1.0:0.20 and 1.0:9.0, respectively, or between about 10% and about 80% by weight of EGCG and about 20% and 90% by weight of caffeine.

In yet another more detailed feature of the invention, the composition is in the form of a pill, tablet, capsule, lozenge, gum, food, oral spray, beverage, toothpaste, powder or other orally administered form or in the form of an absorbent patch.

In yet another more detailed feature of the invention, the method of the present invention comprises administering to the person or other mammal a composition comprising EGCG and caffeine derived from the Camellia sinensis plant in a ratio by weight between 1.0:0.20 and 1.0:9.0, respectively, in an amount sufficient to reduce excess, or maintain healthy, body weight in the person or other mammal.

In yet another more detailed feature of the invention, the method of the invention includes causing a person or other mammal to come into contact with EGCG and caffeine, where the ratio by weight of the EGCG to the caffeine is between 1.0:0.20 and 1.0:4.0, respectively.

In yet another more detailed feature of the invention, the present invention includes an amount of EGCG and caffeine that separately or together come into contact with a person or another mammal, where the ratio by weight of the EGCG to the caffeine is between 1.0:0.20 and 1.0:9.0, respectively.

In yet another more detailed feature of the invention, the EGCG and caffeine of the method and composition are derived from the Camellia sinensis plant.

In yet another more detailed feature of the invention, the step of administering comprises administering approximately 270 milligrams of EGCG and 270 milligrams of caffeine daily.

In yet another more detailed feature of the invention, the step of administering comprises administering the composition daily in three substantially equally divided does, approximately 30 to 60 minutes before meals.

In yet another more detailed feature of the invention, the method of present invention further includes administering to the person or other mammal a composition comprising EGCG and caffeine in a ratio by weight between 1.0:0.20 and 1.0:4.0, respectively, in an amount sufficient to provide energy in an expiatory manner to the person or other mammal.

In yet another more detailed feature of the invention, the method of the present invention further includes identifying at least one person or other mammal that can benefit from an increase in energy.

### DETAILED DESCRIPTION OF THE PREFERRED EMBODIMENTS

The present invention resides in compositions incorporating green tea extract incorporating EGCG, as well as caffeine, in ratios not previously shown. Specifically, EGCG and caffeine are present in the compositions in a preferred ratio of about 1:1. The caffeine in these tea extract compositions is provided is endogenous; i.e., it is caffeine naturally derived from the green tea used to make the extract, instead of being provided from an outside source, whether natural or synthetic. The present invention also resides in methods for controlling body weight, providing immediate expiatory energy, and improving the above-discussed health factors of persons or other mammals, including increasing serum serotonin levels, reducing serum leptin levels, increasing fat oxidation, reducing food intake, and lowering body mass index (BMI). The methods include identifying a person or other mammal who is, or is at risk for being, overweight, or who would benefit either from increased energy levels or the above-described physiological changes, and administering to the person or other mammal a composition comprising sufficient amounts of green tea extract incorporating EGCG along with caffeine, in an amount sufficient to be effective.

As discussed above, the compositions of the present invention incorporate endogenous caffeine, which provides several commercial advantages, including: 1) endogenous green tea caffeine is natural, which is preferred to synthetic sources, 2) it is less expensive and results in a smaller capsule or less capsule fill than adding exogenous natural sources, such as from guarana or gotu-kola, and 3) it provides certain product labeling advantages, including not having to list exogenous sources of caffeine on the ingredients panel. EGCG and caffeine are present in the composition in a ratio of between 1.0:0.20 and 1.0:9.0 more preferably 1.0:0.25 and 1.0:4.0, and most preferably about 1.0:1.0. These ratios are far different from the natural ratios of these components in green tea, in which caffeine levels typically are at least an order of magnitude below EGCG levels. Additionally, a number of published references and available products actually teach that the green tea extract should be decaffeinated, further reducing caffeine levels. EGCG and caffeine preferably are derived from an extract of green tea (Camellia sinensis), though other sources of EGCG and caffeine can be used. In particular, the composition preferably incorporates at least 10% to 80% EGCG and 20% to 90% caffeine, more preferably 30% to 50% each of EGCG and caffeine, and most preferably about 30% each of EGCG and caffeine.

The methods and compositions of the present invention provide for the safe, effective and convenient reduction of excess body weight and resulting reduction in body mass index (BMI), or maintenance of healthy body weight and healthy BMI, in persons or other mammals. Besides these effects, administration of the compositions also provides for reducing serum leptin levels, increasing serum serotonin levels, reducing food intake, increasing fat oxidation, decreasing elevated total and LDL cholesterol, increasing HDL cholesterol, and reducing elevated triglyceride levels in persons or other mammals that would benefit from such effects.

It has been surprisingly found that compositions incorporating EGCG along with a similar or identical amount of caffeine, when administered to persons in sufficient quantities, provide for superior improvement in reducing excess body weight and improving the related health factors described above than was expected, based on the previously know properties of the two components. These compositions also provide for superior energy increase in persons consuming them. Specifically, the combination of EGCG and caffeine in these ratios can reduce body weight, lower body mass index, increase serum serotonin levels, reduce food intake, reduce serum leptin levels, increase fat oxidation, decrease harmful total and LDL cholesterol, increase beneficial HDL cholesterol and lower triglycerides significantly greater than either EGCG alone, or EGCG and caffeine in previously-administered amounts. The composition also provides an expiatory (i.e., immediate) increase in perceived energy level in a person consuming it.

Preferred administration of the composition is orally, in three equally-divided doses roughly 30 to 60 minutes before meals administered daily. For an adult, the preferred composition includes 540 mg of EGCG and caffeine, preferably in a 1.0:1.0 ratio by weight (i.e., 270 mg of EGCG and 270 mg of caffeine). The composition also can include inert ingredients or diluents, such as sugar, maltodextrin, cellulose, or other inert ingredients commonly used in dietary supplements and food and beverage products. The composition may be in various forms, including those commonly used for dietary supplements (e.g., pill, tablet, capsule, lozenge, gum, food, absorbent patch, oral spray, beverage, toothpaste, or powder). The composition also can be incorporated into food or beverage products, including bars, shakes, gums, beverages, or other processed or prepared food or beverage products, or any other orally administerable form. The composition preferably is administered daily in three substantially equally divided doses, approximately 30 to 60 minutes before meals.

Although the invention has been disclosed in detail with reference only to the preferred embodiments, those skilled in the art will appreciate that additional methods and compositions can be made without departing from the scope of the invention.

1. A composition comprising EGCG and caffeine in a ratio by weight between 1.0:0.20 and 1.0:9.0, respectively.

2. A composition as defined in claim 1, wherein the EGCG and caffeine are derived from the Camellia sinensis plant.

3. A composition as defined in claim 1, wherein the EGCG and caffeine are present in a ratio by weight between 1.0:0.25 and 1.0:4.0, respectively.

4. A composition as defined in claim 1, wherein the EGCG and caffeine are present in a ratio by weight of about 1.0:1.0, respectively.

5. A composition as defined in claim 1, wherein the ECGC and caffeine are derived from sources other than the Camellia sinensis plant.

6. A composition as defined in claim 1, wherein the composition is in the form of a pill, tablet, capsule, lozenge, gum, food, oral spray, beverage, toothpaste, powder or other orally administered form.

7. A composition as defined in claim 1, wherein the composition is in the form of an absorbent patch.

8. A composition as defined in claim 1, wherein the composition comprises between about 10% and about 80% by weight of EGCG and about 20% and 90% by weight of caffeine.

9. A composition as defined in claim 8, wherein the composition comprises 30% to 50% by weight of EGCG and 30% to 50% by weight of caffeine.

10. A composition as defined in claim 9, wherein the composition comprises
about 30% by weight of EGCG and about 30% by weight of caffeine.

11. A method for reducing excess, or maintaining healthy, body weight in a person or other mammal comprising:
administering to the person or other mammal a composition comprising EGCG and caffeine derived from the Camellia sinensis plant in a ratio by weight between 1.0:0.20 and 1.0:9.0, respectively, in an amount sufficient to reduce excess, or maintain healthy, body weight in the person or other mammal.

12. A method as defined in claim 11, wherein the EGCG and caffeine are present in a ratio by weight between 1.0:0.25 and 1.0:4.0, respectively.

13. A method as defined in claim 12, wherein the EGCG and caffeine are present in a ratio by weight of about 1.0:1.0, respectively.

14. A method as defined in claim 11, wherein the EGCG and caffeine are derived from sources other than the Camellia sinensis plant.

15. A method as defined in claim 11, wherein the step of administering comprises administering approximately 270 milligrams of EGCG and 270 milligrams of caffeine daily.

16. A method as defined in claim 11, wherein the step of administering comprises administering the composition daily in three substantially equally divided doses, approximately 30 to 60 minutes before meals.

17. A method as defined is claim 16, wherein the step of administering comprises administering the composition orally.

18. A method as defined is claim 11, further comprising identifying a person or other mammal suffering, or at risk of suffering from excess body weight.

19. A method for providing energy in an expiatory manner to a person or other mammal comprising:
administering to the person or other mammal a composition comprising EGCG and caffeine in a ratio by weight between 1.0:0.20 and 1.0:4.0, respectively, in an amount sufficient to provide energy in an expiatory manner to the person or other mammal.

20. A method as defined in claim 19, wherein the EGCG and caffeine are present in a ratio by weight between 1.0:0.25 and 1.0:4.0, respectively.

21. A method as defined in claim 19, wherein the EGCG and caffeine are present in a ratio by weight of about 1.0:1.0, respectively.

22. A method as defined in claim 19, wherein the EGCG and caffeine are derived from the Camellia sinensis plant.

23. A method as defined in claim 19, wherein the step of administering comprises administering approximately 270 milligrams of EGCG and 270 milligrams of caffeine daily.

24. A method as defined in claim 19, wherein the step of administering comprises administering the composition daily in three substantially equally divided doses, approximately 30 to 60 minutes before meals.

25. A method as defined is claim 19, wherein the step of administering comprises administering the composition orally.

26. A method as defined is claim 19, further comprising identifying a person or other mammal that can benefit from an increase in energy.

27. A method for reducing excess, or maintaining healthy, body weight in a person or other mammal comprising:
causing a person or other mammal to come into contact with EGCG and caffeine, wherein the ratio by weight of the EGCG to the caffeine is between 1.0:0.20 and 1.0:4.0, respectively.

28. A method as in claim 27, further comprising providing, either separately or together, EGCG and caffeine.

29. An amount of EGCG and caffeine that separately or together come into contact with a person or another mammal, wherein the ratio by weight of the EGCG to the caffeine is between 1.0:0.20 and 1.0:9.0, respectively.
